(19) United States
(12) Patent Application Publication
(10) Pub. No.: US 2026/0007473 A1
(43) Pub. Date: Jan. 8, 2026

(54) Hybrid Anatomical Registration of a Body

(71) Applicant: INTELLIJOINT SURGICAL INC., KITCHENER (CA)

(72) Inventors: JOSEPH ARTHUR SCHIPPER, Kitchener (CA); Andre Novomir Hladio, Waterloo (CA)

(21) Appl. No.: 18/761,715

(22) Filed: Jul. 2, 2024

(51) Int. Cl. A61B 34/20; A61B 17/17; A61B 34/10; A61B 34/30; A61B 90/00

(52) U.S. Cl. CPC A61B 34/20; A61B 17/1703; A61B 2034/107; A61B 2034/2055; A61B 2034/2065; A61B 34/30; A61B 2090/364; A61B 2090/3762

(57) ABSTRACT

Systems and methods for hybrid registration of a portion of patient anatomy facilitate surgical navigation. A navigation system track objects comprising any one or more of surgical tools and portions of patient anatomy and receives a first subset of anatomical reference information for a portion via at least one intra-operative image. Each image has a common image reference frame and shows a same portion of patient anatomy. The image reference frame is registered with a navigation system reference frame. One or more navigational system sensors provide signals identifying a second subset of anatomical reference information for the portion of anatomy, different from the first subset. The portion of patient anatomy is registered to the navigation system using at least one item of anatomical reference information from each of the first subset of anatomical reference information and the second subset of anatomical reference information for performing navigation of the patient anatomy

### FIELD OF INVENTION

The present application relates to surgical navigation systems and more particularly to systems and methods for use in registration of a portion of a patient's anatomy for use during surgery.

### BACKGROUND

In orthopaedic surgery, such as hip and knee surgery, it is desirable to perform an “anatomical registration”, wherein a portion of a patient is registered to a navigational system in order to assist in a surgery. Often the portion of the patient is represented or characterized by one or more patient landmarks and/or axes, etc. associated with the portion of the patient. A registration operation computes a mapping or correlation between a reference frame of the navigational system and a reference frame of the portion of the patient. A known method to register patient anatomy includes imageless registration, wherein the navigational system does not rely on images of the patient anatomy, per se, such as from an intra-operative imaging device, to register anatomical landmarks. An example of imageless registration operations includes tracking a device (such as a probe) and identifying anatomical landmarks to the navigational system using the tracked device.

It often desirable to utilize one or both of intra-operative images (either two dimensional (2D) or three dimensional (3D) images) and pre-operative images (usually 3D images) during a surgery because of their wealth of information. Examples of intra-operative images and pre-operative images include: x-ray images, CT scan images, and ultrasound images, among other modalities. It is known to correlate these intra-operative images and pre-operative images, i.e. their respective reference frames, using image to image registration.

It is further known to register a reference frame of intra-operative images or a reference frame of pre-operative images, or both, to the reference frame of the navigational system. Intra-operative or pre-operative images are useful for performing anatomical registration using image based registration techniques. Image based registration allows for anatomical landmarks to be acquired through the skin, allowing for inaccessible landmarks to be used for registration and the registration to occur either fully or partially prior to performing a primary incision of a surgery.

Despite this, intra-operative image-based registration does not work well for at least some orthopaedic applications, such as where the anatomical registration may require landmarks that fall outside the field of view of an image generated by an intra-operative image device. As an example, an intra-operative fluoroscopic imager may only have a 12″ imaging detector limit, whereas a human femur may be 20″ long. Additionally, capturing the entire portion of the anatomy to be registered within a single image (or even a series of images) may be unfeasible. There is often an inability to manipulate an imager (such as a c-arm fluoroscopic imager) to the required locations to image the entire portion as operating table structures can impede the movement of the imager. Registering to a pre-operative image is also infeasible for most practical applications. Pre-operative images are often obtained weeks prior to the surgical procedure and may not represent current state of anatomy.

It is therefore desirable to provide improved methods and systems for surgical navigation which can both take advantage of the benefits of existing registration methods while simultaneously overcoming the weaknesses of each one.

### SUMMARY

Systems and methods for hybrid registration of a portion of patient anatomy facilitate surgical navigation. A navigation system track objects comprising any one or more of surgical tools and portions of patient anatomy and receives a first subset of anatomical reference information for a portion via at least one intra-operative image. Each image has a common image reference frame and shows a same portion of patient anatomy. The image reference frame is registered with a navigation system reference frame. One or more navigational system sensors provide signals identifying a second subset of anatomical reference information for the portion of anatomy, different from the first subset. The portion of patient anatomy is registered to the navigation system using at least one item of anatomical reference information from each of the first subset of anatomical reference information and the second subset of anatomical reference information for performing navigation of the patient anatomy

There is provided a computer-implemented method for surgical navigation, comprising executing by a processor the steps of: receiving an identification of a first subset of anatomical reference information associated with at least one intra-operative image of a surgical location, each intra-operative image having an image reference frame and each showing a same portion of patient anatomy; registering the image reference frame with a navigation system reference frame of a navigation system, wherein the navigation system is configured to track objects comprising any one or more of surgical tools and portions of patient anatomy; receiving signals, from one or more navigational system sensors, identifying a second subset of anatomical reference information, wherein at least some of the second subset of anatomical reference information are different from the first subset of anatomical reference information; and registering a portion of patient anatomy to the navigation system using at least one item of anatomical reference information from each of the first subset of anatomical reference information and the second subset of anatomical reference information for performing navigation of the patient anatomy.

There is provided a computer system where the system is configured to perform a computer-implemented method herein.

### BRIEF DESCRIPTION OF DRAWINGS

FIG. 1 shows components of an environment in which hybrid anatomical registration may be practised.

FIG. 2 is a flowchart of a computer-implemented method in accordance with an embodiment.

### DETAILED DESCRIPTION

Systems, methods and devices are presented herein pertaining to hybrid anatomical registration of a body for surgical navigation. The embodiments refer to types of surgeries, including knee and hip surgery. However, a person skilled in the art will realize that the specification is applicable to other forms of surgery and is not meant to be limited to those described. It is further understood that various methods described for performance by a computer system such as navigational surgery may be implemented in software such as instructions and data to configure at least one processing unit of a computer system to perform the method. The instructions and data may be stored in a device such as a memory (RAM, ROM, flash drive, etc.) or other non-transitory storage device (e.g.: magnetic, optical, or other disk or storage medium).

Several systems, methods and devices will be described below as embodiments. The scope of the claims should not be limited by the embodiments set forth in the examples but should be given the broadest interpretation consistent with the description as a whole.

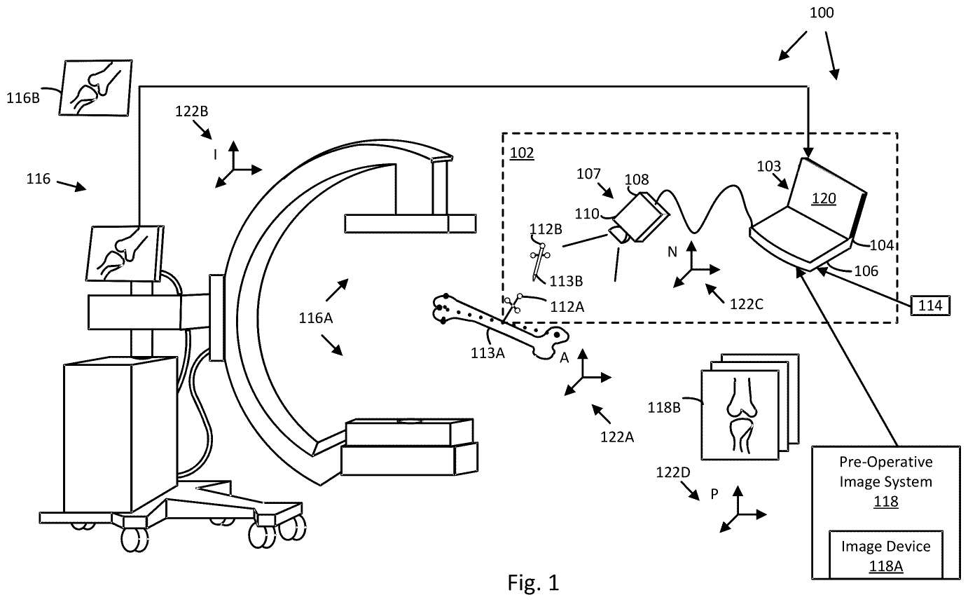

Referring now to FIG. 1, there is shown components of an environment 100, in accordance with an embodiment, in which hybrid anatomical registration may be practised according to the teachings herein. It will be apparent that some components are optional as further described.

In the environment 100, a surgical navigation system 102 (generally denoted by the dashed line rectangle) comprises a computing device 103 including a central processing unit (CPU) 104 and a storage device 106, such as memory and persistent storage devices, etc. The surgical navigation system 102 also comprises an optical sensor unit 107 having at least one navigation system sensor. In the illustrated embodiment, the at least one navigation system sensor comprises an optical sensor 108 (e.g. in the form of a tracking camera) and at least one other sensor 110. In an embodiment, the optical sensor unit is configured for attachment to an object such as a patient anatomy, an operating table, or a surgical tool (all not shown). The optical sensor unit (e.g. its tracking camera 108 and/or at least one sensor 110) can be coupled for communication to the computing device 103 for communicating sensor signals, for example, for optical sensor 108, such signals comprise images. Examples of the at least one sensor 110 can be a gyroscope, an accelerometer, and/or a magnetometer. In an embodiment, the at least one sensor 110 is not present and the surgical navigation system receives optical signals such as for tracking an object.

Although the illustrated embodiment shows an optical-based tracking capability, another example of a navigation system tracking technology comprises electromagnetic (EM) tracking using electromagnetic field generation around a portion of the patient. Optical and electromagnetic sensor technologies can be combined.

Surgical navigation system 102 comprises an optically detectable target (e.g. 112A and 112B). The targets 112A and 112B are configured for coupling to (or forming part of) an object 113A or 113B such as a trackable tool, implant, or patient anatomy, etc. The tracked object typically does not form a component of the system 102, particularly when the object (e.g. 113A) is a portion of a patient such as a femur. Target 112A coupled to femur 113A is shown in a representative manner for simplified illustration purposes and not necessarily coupled at a preferred location for conducting a navigational surgical procedure. Examples of the trackable tool for assisting with a registration operation include a probe (e.g. 113B as shown) or an axes frame (not shown). In an embodiment, the probe 113B is manufactured with a permanent target that forms a part of the probe. An example of optically detectable targets are disclosed in publication US2022/0332972 A1 dated Oct. 20, 2022 entitled, “Systems, Targets and Methods for Optical Registration of Tools”, the entire contents of which are incorporated herein by reference. Though not shown, a tool can be tracked (wholly or partially) through its inherent features (e.g. natural visual features) such that a tacker for coupling to the tool is not necessary. An example of natural feature tracking is described in US2020/0305986 A1, dated Oct. 1, 2020, the entire contents of which are incorporated herein by reference.

Representative optical sensors and trackers have been disclosed in Applicant's U.S. Pat. No. 9,247,998, granted Feb. 2, 2016, and entitled “System and Method of Intra-Operative Leg Position Measurement”, and U.S. Pat. No. 9,603,671, granted Mar. 28, 2017, and entitled, “Systems, methods and devices for anatomical registration and surgical localization” the contents of each of which are incorporated herein by reference in their respective entireties. A method and system for surgical navigation has been disclosed in Applicant's U.S. Pat. No. 9,247,998, granted Feb. 2, 2016, and entitled “System and Method of Intra-Operative Leg Position Measurement”, the contents of which is incorporated herein by reference in its entirety. A further example of an embodiment of a surgical navigation system is shown in publication US2021/0121237 A1 dated Apr. 29, 2021 and entitled, “Systems and Methods for Augmented Reality Display in Navigated Surgeries”, the contents of which are incorporated herein by reference.

In an embodiment, the optically detectable target 112A/112B provides optical signals to the tracking camera 108. The optical signals received by the tracking camera 108 are provided to the navigation system 102 and processed by the CPU 104. In another configuration, the at least one navigation system sensor 110 provides signals to the navigation system 102 which are processed by the CPU 104. In other embodiments (not shown), electromagnetic signals are provided for tracking purposes.

The optical sensor unit 107 may be configured in accordance with one of the following: a) multi-spectral camera (providing visible and tracking channels); (b) dual cameras (providing respective visible and tracking channels); (c) dual imager (using prism to split visible and tracking channels); or (d) tracking channel using visible light; among other configurations.

In the environment 100, the navigation system 102 is shown configured to receive input 114, such as in the form of signals delivered via a keyboard, mouse, or voice command. In the environment, 100, navigation system 102 is coupled for (data) communication with an intra-operative image system 116, and a pre-operative image system 118. The coupling can be via wireless or wired means, for example. Though not shown, data (e.g. from a pre-operative image system 118) can be provided via other means such as a thumb drive, CD-ROM, or other storage device. In an embodiment, pre-operative image data is pre-loaded to storage 106, for example, prior to a surgery.

Intra-operative image system 116 is configured with an intra-operative image device 116A in accordance with the imaging modality provided by system 116. Intra-operative image system 116 provides intra-operative image data 116B, for example comprising one or more intra-operative images, to navigation system 102. Examples of intra-operative image systems include: Fluoroscopy-based systems, Cone-Beam computed tomography (CT)-based systems, and Fan Beam CT-based systems, among others. In an embodiment, such as illustrated. The optical sensor 108 providing optical signals to computing device 103 is different from (e.g. separate from and a distinct component from) the intra-operative image device 116A.

Pre-operative image system 118 is configured with an image device 118A. Examples of a pre-operative image systems include: X-Ray-based systems, CT-based systems, and/or magnetic resonance imaging (MRI) based systems, among other modalities. Intra-operative image data comprises: surgical landmark data, image data of a patient anatomy, and image reference frame data. Pre-operative image data comprises: surgical landmark data, image data of a patient anatomy, and image reference frame data.

FIG. 1 further illustrates a plurality of respective reference frames 122A, 122B, 122C and 112D for each of: the surgical site (e.g. 122A), the intra-operative image system 116 (e.g. 122B), the navigation system (e.g. 122C) and the pre-operative images system 118 (e.g. 122D). It will be appreciated that the data/information from (or within) each of these environments is associated with the respective reference frame. Registration operations, for example, can be performed by navigation system 102 to construct applicable mappings to relate one reference frame with another, for example, to facilitate usage of data from another environment within the environment of navigation system 102.

As a result of the different imaging or other data sensing modalities used, for example, intra-operative image systems (e.g. 116) and navigational systems (e.g. 102) have respective strengths and weaknesses in relation to identifying certain types of information. For example, and without limitation, intra-operative image systems can produce images (e.g. 116B) having richer and/or more accurate information about subcutaneous anatomical landmarks. As many surgical procedures follow minimally invasive techniques, only small surgical volumes are opened. Some anatomical landmarks that are useful for navigational surgery such as to register a portion of the patient's anatomy, are often not exposed during a procedure. Image modalities that illuminate subcutaneous structures provide information that is not necessarily available to a navigation system using typical tracking techniques such as identifying a landmark using a probe. Often navigation systems are stronger at tracking motion of a patient anatomy, for example, when rotating about a joint, or stronger at tracking/identifying landmarks in a larger field of view or by moving the optical sensor of the navigation system to obtain a different field of view.

Furthermore, intra-operative image devices, such as device 116A, typically has a defined field of view that is relatively small and, once positioned, the intra-operative image system 116 is generally stationary during a surgical procedure. As such, the images 116B comprise a relatively small portion of patient anatomy and each image 116B includes a same subset of patient anatomical landmarks. As such successive images from image device 116A do not provide additional reference information about additional landmarks.

Though not shown, navigation system 102 can be configured to receive, store, and present pre-operatively generated planning data such as from a planning computer system (not shown), which is often presented in association with pre-operative images 118B. Operations can register the reference frame of the pre-operative images to the reference frame of the navigation system, such as using known techniques.

In an embodiment, navigation system 102 is configured to provide information to a display 120 to provide navigational guidance during a surgery, including imaging data which can be derived from intra-operative data and/or pre-operative image data, overlay data (e.g. augmented reality data), measurement data, and workflow such as instructions to assist with performing a procedure displayed in a graphical user interface, etc.

In an embodiment, navigation system 102 is configured to provide navigation in association with a portion of a patient's anatomy during a surgery. To do so, registration operations are performed to enable the navigation system 102 to work with the portion of the patient's anatomy.

Generally, in order to register the portion of the patient's anatomy, the navigation system 102 is provided with reference information, typically anatomical information, to construct a mapping (i.e. a registration) between a reference frame of the navigation system and a real-world reference frame in a space where the portion of the patient's anatomy is located. It will be understood that anatomical reference information comprises data related to an anatomical landmark, an anatomical axis, etc. It will be understood that an anatomical landmark could represent a point in space (typically on a surface of the portion of the anatomy), or a region in space. A region can be a volume, for example, a portion of a surface of a patient.

In an embodiment, there is more than one source of reference information with which the mapping can be defined. For example, anatomical reference information may find its source in images (e.g. 116B) from an intra-operative image system 116, tracking information from the navigation system sensors, and, optionally, images (e.g. 118B) from a pre-operative image system 116 such as further described herein.

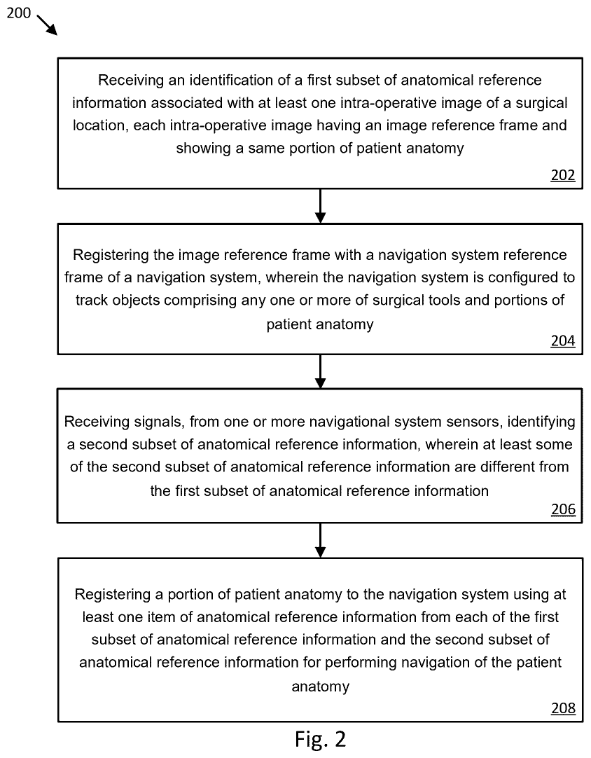

FIG. 2 is a flowchart showing operations 200 of a computing device, such as device 103 for performing a computer implemented method according to an embodiment. In an embodiment, a computer implemented method comprises executing by a processor one or more (method) steps.

At 202, operations (e.g. of the surgical navigation system 102) receive an identification of a first subset of anatomical reference information associated with at least one intra-operative image of a surgical location, each intra-operative image having an image reference frame (e.g. in the form of image reference frame data) and each showing a same portion of patient anatomy. The first subset can be received from an intra-operative image device (e.g. 116A). The first subset can comprise a plurality of items of information such as an axis or axes, and anatomical landmark or landmarks, etc.

In an embodiment, the first subset of anatomical reference information is represented in the image 116B. This anatomical reference information is identified to navigation system 102. In an embodiment, the image can be presented, such as via display 120, to a human operator and the operator can provide input (e.g. 104) to identify one or more landmarks represented in the image. The input can comprise a click or other gesture, etc. at the location of the landmark(s). In an embodiment, the landmarks can be identified automatically, without operator location input. For example, image processing using artificial intelligence (AI) or other techniques can identify the landmarks. One or more deep neural networks (DNNs) configured for image processing can be trained, for example, using supervised learning (or other learning models) to localize the one or more landmarks expected to be present in the image 116B. The one or more DNNs may be trained to classify the presence of the landmarks, for example, providing a result indicating whether a particular landmark is or is not located. In an example, the one or more DNNs performs segmentation of the image 116B, for example, defining a mask that identifies the pixels where the landmark is located.

Workflow and other operations of navigation system 102 can be responsive to the output of the identification either by human input or a landmark localization engine comprising the one or more DNNs. For example, additional anatomical reference information can be calculated from the location of the landmarks. In an embodiment, a clinically relevant axis can be determined using two or more landmarks. Further, in an embodiment, a rendering pipeline can render an effect such as over image 116B for display via display 120, which shows the location of the landmarks and/or other determined reference information.

At 204, operations register the image reference frame with a navigation system reference frame of the navigation system 102, wherein the navigation system 102 is configured to track objects comprising any one or more of surgical tools and one or more portions of patient anatomy.

In some configurations, the registration of the image reference frame to the navigation system reference frame occurs through the use of fiducials. The fiducials are present in both the intra-operative image and a different image available to the navigation system 102 such as through the optical sensor 108. It is common (but not necessary) for image fiducials and navigation fiducials to be different fiducials, but having a known registration or positional relationship. A mapping for the registration can be determined through processing the images based on the locations of the fiducials. Human input can identify the respective fiducials such as discussed with reference to identifying anatomical information hereinabove. Other processing techniques may be used such as image processing techniques using a trained model (e.g. as per the one or more Deep Neural Networks described hereinabove) to identify fiducials in an image. Such processing can process each image (e.g. the image from the system 116 and the image from the navigation system 102) to identify the same fiducials in each for performing the registration of the reference frames.

At 206, operations receive signals, (e.g. from one or more navigational system sensors), identifying a second subset of anatomical reference information, wherein at least some items of the second subset of anatomical reference information are different from items of the first set of anatomical reference information. The second subset can comprise a plurality of items of information such as an axis or axis, an anatomical landmark or landmarks, etc.

Signals from the navigational system sensors can include optical signals. The optical signals can include information from a tracker 112B coupled to a trackable tool. In an alternative example, tracker 112 is coupled to a portion of patient anatomy, e.g. the portion desired to be registered in the navigation system. In an example, the position of the tracker identifies the second subset of anatomical reference information such as anatomical landmarks. Processing the information from the tracker identifies the second subset of anatomical landmarks for the navigation system 102. Processing operations can identify landmarks or other reference information such as: 1) by defining points using a tracker, for example, a tracker coupled to a probe whose tip location is measured by the navigation system; or 2) via calculation of a point or axis, for example, by attaching the tracker to the anatomy and moving the anatomy to compute a center of rotation of a joint.

At 208, operations register a portion of patient anatomy to the navigation system using at least one item of anatomical reference information from each of the first subset and the second subset for performing navigation of the patient anatomy.

With the anatomical reference information from the different sources now registered in a common reference frame of the navigation system 102 (the navigation system reference frame), the navigation system 102 can provide surgical navigation in relation thereto.

By doing so, the navigation system 102 performs surgical navigation of the patient anatomy using the portion of patient anatomy as registered.

In an example, performing surgical navigation can provide clinically relevant information for one of a knee arthroplasty or a hip arthroplasty, for example, where the portion of patient anatomy comprises a pelvis, a femur or a tibia, as is applicable.

In another example, the navigation system 102 receives the intra-operative image in the form of intra-operative image data, comprising: surgical landmark data, image data of a patient anatomy, and image reference frame data.

As previously noted, the optical sensor 108 and the intra-operative image device 116A of the intra-operative image system 116 are different from one another as separate and distinct apparatus. Through the use of an intra-operative image device 116A and the optical sensor 108, the navigation system 102 receives image data from a field of view that can be wider than either the optical sensor 108 or the intra-operative image camera can individually capture on their own.

It will be appreciated that the at least one item of anatomical reference information from each of the first subset and the second subset can comprise clinically relevant information to define the portion of the patient anatomy.

Defining clinically relevant information is important as in many implant-related surgeries, clinically relevant information in the form of measurements are required to ensure implant positions align with those planned for in the pre-operative phase.

In an example, the at least one item of anatomical reference information from the first subset used to perform the registration is different from the at least one item of anatomical reference information of the second subset used to perform the registration. By doing so, the navigational system can register a portion of a patient anatomy using anatomical reference information located from one system (e.g. the intra-operative image system 116) that is not available via the other system (e.g. the sensors 108/110 of the navigation system 102) or vice versa.

In another example, the navigation system 102 registers the portion of patient anatomy to the navigation system further using at least one item of anatomical reference information from a third subset of anatomical reference information in the form of pre-operative image data derived from a pre-operative image from the pre-operative image system 118. In an example, the at least one item of anatomical reference information from the third subset is different from the at least one item of anatomical reference information from each of the first subset and the second subset. By doing so, the navigational system can register a portion of a patient anatomy using anatomical reference information located from one system (e.g. the intra-operative image system 116, or pre-operative image system 118) that is not available via the other system (e.g. the sensors 108/110 of the navigation system 102), for example.

In another example, the navigation system 102 registers the pre-operative image to the navigation system 102 using an image reference frame of the pre-operative image in the form of image reference frame data. The pre-operative image can be registered to the navigation system in different ways, including through the identification of some commonalities, such as fiducials or landmarks, between the pre-operative image data and the navigation system 102, and registering the pre-operative image directly to the navigation system 102. In another example, the navigation system 102 registers the pre-operative image to the navigation system 102 via the intra-operative image using image-to-image registration. In such image-to-image registration, common features in images from each system are identified and a mapping (e.g. a registration) is constructed such that information in one reference frame is relatable to information in the other reference frame, as is known. For example, commonalities between the pre-operative image and intra-operative image, such as fiducials and landmarks, are identified, and the pre-operative image is mapped onto the image reference frame of the intra-operative image.

In another example, the navigation system 102 registers the portion of patient prior to an occurrence of a primary incision during a surgery.

In many surgeries, particularly orthopaedic surgeries such as a hip arthroplasty or knee arthroplasty, procedures in the form of minimally invasive surgeries are followed. When a minimally invasive surgery procedure is followed, a small portion of patient anatomy is opened up, with the aim of minimizing the small portion of patient anatomy opened up for various reasons, including: to avoid infection, reduce operating time, minimize blood loss, etc. This opening up of the small portion of patient anatomy is the primary incision. Prior to the primary incision, various surgeries and forms of registrations may first require one or more smaller incisions into portions of patient anatomy. Examples of the one or more smaller incisions include incisions to mount trackers onto a patient (for example through a bone screw mounted to a bone).

Techniques herein are useful in a knee surgery, for example. Patient anatomy data shown in the at least one intra-operative image can show a knee region. The first subset of anatomical reference information can comprise information related to the knee, for example, femur information such as the lateral and medial condyles, the transepicondylar axis, Whiteside's line, posterior condylar axis, etc. or proximal tibia information for defining the center of the tibia at the knee, the tibial anterior-posterior axis, the medial third tubercle, native tibial slope, etc. depending on the portion of the anatomy to be registered. The second subset of anatomical reference information from the one or more sensors of the navigation system 102 can comprises either: a hip center of rotation to register a femur as the portion of the patient anatomy; or both malleoli (for determining a centre of the distal tibia at the ankle) to register a tibia as the portion of the patient anatomy.

Registering a femur using a hip centre of rotation can be performed by (rigidly) coupling a tracker to the femur and using the tracking capability of system 102 such as optical sensor 108 to collect optical signals from the tracker. As the femur coupled to the tracker is rotated and the tracker rotates with the femur, the optical sensor 108 gathers tracking data with which to observe the femur being rotated around it's (generally) stationary end-point located in the hip joint. The navigation system 102 can determine the hip centre of rotation (HCOR) in the acetabulum. The HCOR is useful to determine a mechanical axis of the femur when the first subset of anatomical reference information includes data for determining a center at the distal femur.

In another example, the patient anatomy data in the at least one intra-operative image includes intra-operative image data in the form of image data of a patient anatomy, specifically a hip region, including a proximal portion of a femur. In this same example, the second subset of anatomical landmarks defines clinically relevant landmarks of a distal portion of the femur.

In furtherance to this example, the proximal portion of the femur is identified by the navigation system 102 through a landmark in the first subset of anatomical landmarks provided to the navigation system 102 by the intra-operative image system 116. The navigation system 102 can then provide a mechanical axis between the femoral head center (which is located in the acetabulum and the distal end of the femur, the mechanical axis being clinically relevant information to a number of surgeries, such as total knee arthroplasty.

In an example, the signals received from the one or more navigational system sensors of the navigational system 102 are received from at least one sensor 110, the at least one sensor 110 being a separate component from the optical sensor 108.

In an example, the at least one sensor 110 of the navigation system 102 comprises one or more of: an optical sensor, an accelerometer, a gyroscope, and a magnetometer.

In an example, the navigation system 102 provides to a display 120 any one or more of: a relative measurement between the tool and the patient's anatomical structure; an overlay for a portion of the tool or the cutting plane for display with an image of the patient's anatomical structure; workflow for a procedure; and an instruction to command a robotic manipulator.

In an embodiment, a planar volumetric cutting tool is tracked, for example, relative to a bone of a patient's anatomy. A surgical navigation system can perform relative measurements between the tool and the bone. One example measurement is a resection depth determined relative to a portion of the bone, and, for example, a cutting plane provided by one of the cutting surfaces of the blade of the tool. In an embodiment, a graphical user interface (GUI) may present the measurements. In an embodiment, an overlay representing the cutting surface can be defined from the tracked tool and its pose updated in response to the pose of the tool. The overlay can be presented in association with an image of the bone (e.g. a real-time image) via a display device, such as part of an augmented reality GUI. In some of the embodiments herein, the surgical navigation system is configured to provide instructions to configure the tool such as with a tracker on a mounting adapter. In an embodiment, the GUI or other interface is configured to receive user input such as to provide tool and/or tracker configuration information, for example, identifying data about the geometry of any one or more of the tool, the tracker, any mounting information, etc. In any of the embodiments herein, the surgical navigation system can be configured to present workflow for a particular procedure, such as a TKA procedure.

It will be understood that the surgical navigation system can, in respective embodiments, be configured to provide guidance information such as measurements, commands, etc. to a GUI, a storage device such as a memory or, via communication, to another system such as a robot manipulator to manipulate the positon of the tool. In an embodiment, the planar volumetric cutting tool is configured to be hand-held, used with a cutting guide, mounted on an arm that is manually controlled, mounted on an arm that is robotically controlled and any combination thereof.

In addition to computing device and method aspects, a person of ordinary skill will understand that computer program product aspects are disclosed, where instructions are stored in a non-transient storage device (e.g. a memory, CD-ROM, DVD-ROM, disc, etc.) and that, when executed, the instructions cause a computing device to perform any of the method aspects stored herein.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers characteristics, compounds, chemical moieties or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

In accordance with embodiments herein, there is provided at least one method, system or apparatus as set for the in the following numbered “Statements” including:

Statement 1: A computer-implemented method for surgical navigation, comprising executing by a processor the steps of:
receiving an identification of a first subset of anatomical reference information associate with at least one intra-operative image of a surgical location, each intra-operative image having an image reference frame and each showing a same portion of patient anatomy;
registering the image reference frame with a navigation system reference frame of a navigation system, wherein the navigation system is configured to track objects comprising any one or more of surgical tools and portions of patient anatomy;
receiving signals, from one or more navigational system sensors, identifying a second subset of anatomical reference information, wherein at least some of the second subset of anatomical reference information are different from the first subset of anatomical reference information; and
registering a portion of patient anatomy to the navigation system using at least one item of anatomical reference information from each of the first subset of anatomical reference information and the second subset of anatomical reference information for performing navigation of the patient anatomy.

Statement 2: The computer-implemented method of Statement 1 comprising performing a surgical navigation of the patient anatomy using the portion of patient anatomy as registered.

Statement 3: The computer-implemented method of Statement 2, wherein performing the surgical navigation provides clinically relevant information for one of a knee arthroplasty or a hip arthroplasty.

Statement 4: The computer-implemented method of any preceding Statement comprising receiving the intra-operative image.

Statement 5: The computer-implemented method of any preceding Statement, wherein the one or more navigational system sensors comprises an optical sensor; and, each intra-operative image is generated by an intra-operative image device that is different from the optical sensor.

Statement 6: The computer-implemented method of any preceding Statement, wherein the one or more navigational system sensors comprises one or more of: an optical sensor; an accelerometer; a gyroscope; a magnetometer; or an electromagnetic field sensor.

Statement 7: The computer-implemented method of any preceding Statement, wherein the at least one item of anatomical reference information from each of the first subset and the second subset comprises clinically relevant information to define the portion of the patient anatomy.

Statement 8: The computer-implemented method of any preceding Statement, wherein the at least one item of anatomical reference information from the first subset used to perform the registration is different from the at least one anatomical reference information of the second subset used to perform the registration.

Statement 9: The computer-implemented method of any preceding Statement comprising registering a pre-operative image to the navigation system using an image reference frame of the pre-operative image and providing surgical navigation in association with the pre-operative image and a surgical plan.

Statement 10: The computer-implemented method of any preceding Statement, wherein the portion of patient anatomy is registered prior to an occurrence of a primary incision during a surgery.

Statement 11: The computer-implemented method of any preceding Statement, wherein the patient anatomy shown in the at least one intra-operative image shows a knee region and the second subset of anatomical reference information comprises either:
a hip center of rotation to register a femur as the portion of the patient anatomy; or
both malleoli to register a tibia as the portion of the patient anatomy.

Statement 12: The computer-implemented method of any preceding Statement, wherein the patient anatomy shown in the at least one intra-operative image shows a hip region, including a proximal portion of a femur, and the second subset of anatomical reference information defines clinically relevant landmarks of a distal portion of the femur.

Statement 13: The computer-implemented method of any preceding Statement comprising providing any one or more of:
a relative measurement between the tool and the patient's anatomical structure;
an overlay for a portion of the tool or the cutting plane for display with an image of the patient's anatomical structure;
workflow for a procedure; or
an instruction to command a robotic manipulator.

Statement 14: The computer-implemented method of any preceding Statement, wherein anatomical reference information comprises one or more of anatomical landmark information and anatomical axis information.

Statement 15: The computer-implemented method of any preceding Statement, wherein the at least one image comprises one of: a fluoroscopy-based image, a one-Beam computed tomography (CT)-based image, or a Fan Beam CT-based image.

Statement 16: A navigation system comprising at least one processor and at least one storage device storing executable instructions that when executed by the at least one processor cause the navigation system to:
receive an identification of a first subset of anatomical reference information associate with at least one intra-operative image of a surgical location, each intra-operative image having an image reference frame and each showing a same portion of patient anatomy;
register the image reference frame with a navigation system reference frame of the navigation system, wherein the navigation system is configured to track objects comprising any one or more of surgical tools and portions of patient anatomy;
receive signals, from one or more navigational system sensors, identifying a second subset of anatomical reference information, wherein at least some of the second subset of anatomical reference information are different from the first subset of anatomical reference information; and
register a portion of patient anatomy to the navigation system using at least one item of anatomical reference information from each of the first subset of anatomical reference information and the second subset of anatomical reference information for performing navigation of the patient anatomy.

Statement 17: The navigation system of claim 16, wherein the execution of the instructions further causes the system to perform a surgical navigation of the patient anatomy using the portion of patient anatomy as registered, wherein performing the surgical navigation provides clinically relevant information for a surgery procedure.

Statement 18: The navigation system of claim 16 or 17, wherein the one or more navigational system sensors comprises an optical sensor; and, each intra-operative image is generated by an intra-operative image device that is different from the optical sensor.

Statement 19: The navigation system of claim 16, 17 or 18, wherein the execution of the instructions further causes the system to provide any one or more of:
a relative measurement between the tool and the patient's anatomical structure;
an overlay for a portion of the tool or the cutting plane for display with an image of the patient's anatomical structure;
workflow for a procedure; or
an instruction to command a robotic manipulator.

Statement 20: A computer program product comprising a non-transitory storage device storing instructions for execution by at least one processor of a navigation system to cause the navigation system to: receive an identification of a first subset of anatomical reference information associate with at least one intra-operative image of a surgical location, each intra-operative image having an image reference frame and each showing a same portion of patient anatomy; register the image reference frame with a navigation system reference frame of the navigation system, wherein the navigation system is configured to track objects comprising any one or more of surgical tools and portions of patient anatomy; receive signals, from one or more navigational system sensors, identifying a second subset of anatomical reference information, wherein at least some of the second subset of anatomical reference information are different from the first subset of anatomical reference information; and register a portion of patient anatomy to the navigation system using at least one item of anatomical reference information from each of the first subset of anatomical reference information and the second subset of anatomical reference information for performing navigation of the patient anatomy.

It will be appreciated that system Statements and apparatus Statements corresponding to any method Statement are contemplated herein.

What is claimed is:

1. A computer-implemented method for surgical navigation, comprising executing by a processor the steps of:
receiving an identification of a first subset of anatomical reference information associate with at least one intra-operative image of a surgical location, each intra-operative image having an image reference frame and each showing a same portion of patient anatomy;
registering the image reference frame with a navigation system reference frame of a navigation system, wherein the navigation system is configured to track objects comprising any one or more of surgical tools and portions of patient anatomy;
receiving signals, from one or more navigational system sensors, identifying a second subset of anatomical reference information, wherein at least some of the second subset of anatomical reference information are different from the first subset of anatomical reference information; and
registering a portion of patient anatomy to the navigation system using at least one item of anatomical reference information from each of the first subset of anatomical reference information and the second subset of anatomical reference information for performing navigation of the patient anatomy.

2. The computer-implemented method of claim 1 comprising performing a surgical navigation of the patient anatomy using the portion of patient anatomy as registered.

3. The computer-implemented method of claim 2, wherein performing the surgical navigation provides clinically relevant information for one of a knee arthroplasty or a hip arthroplasty.

4. The computer-implemented method of claim 1 comprising receiving the intra-operative image.

5. The computer-implemented method of claim 1, wherein the one or more navigational system sensors comprises an optical sensor; and, each intra-operative image is generated by an intra-operative image device that is different from the optical sensor.

6. The computer-implemented method of claim 1, wherein the one or more navigational system sensors comprises one or more of: an optical sensor; an accelerometer; a gyroscope; a magnetometer; or an electromagnetic field sensor.

7. The computer-implemented method of claim 1, wherein the at least one item of anatomical reference information from each of the first subset and the second subset comprises clinically relevant information to define the portion of the patient anatomy.

8. The computer-implemented method of claim 1, wherein the at least one item of anatomical reference information from the first subset used to perform the registration is different from the at least one anatomical reference information of the second subset used to perform the registration.

9. The computer-implemented method of claim 1 comprising registering a pre-operative image to the navigation system using an image reference frame of the pre-operative image and providing surgical navigation in association with the pre-operative image and a surgical plan.

10. The computer-implemented method of claim 1, wherein the portion of patient anatomy is registered prior to an occurrence of a primary incision during a surgery.

11. The computer-implemented method of claim 1, wherein the patient anatomy shown in the at least one intra-operative image shows a knee region and the second subset of anatomical reference information comprises either:
a hip center of rotation to register a femur as the portion of the patient anatomy; or
both malleoli to register a tibia as the portion of the patient anatomy.

12. The computer-implemented method of claim 1, wherein the patient anatomy shown in the at least one intra-operative image shows a hip region, including a proximal portion of a femur, and the second subset of anatomical reference information defines clinically relevant landmarks of a distal portion of the femur.

13. The computer-implemented method of claim 1 comprising providing any one or more of:
a relative measurement between the tool and the patient's anatomical structure;
an overlay for a portion of the tool or the cutting plane for display with an image of the patient's anatomical structure;
workflow for a procedure; or
an instruction to command a robotic manipulator.

14. The computer-implemented method of claim 1, wherein anatomical reference information comprises one or more of anatomical landmark information and anatomical axis information.

15. The computer-implemented method of claim 1, wherein the at least one image comprises one of: a fluoroscopy-based image, a one-Beam computed tomography (CT)-based image, or a Fan Beam CT-based image.

16. A navigation system comprising at least one processor and at least one storage device storing executable instructions that when executed by the at least one processor cause the navigation system to:
receive an identification of a first subset of anatomical reference information associate with at least one intra-operative image of a surgical location, each intra-operative image having an image reference frame and each showing a same portion of patient anatomy;
register the image reference frame with a navigation system reference frame of the navigation system, wherein the navigation system is configured to track objects comprising any one or more of surgical tools and portions of patient anatomy;
receive signals, from one or more navigational system sensors, identifying a second subset of anatomical reference information, wherein at least some of the second subset of anatomical reference information are different from the first subset of anatomical reference information; and
register a portion of patient anatomy to the navigation system using at least one item of anatomical reference information from each of the first subset of anatomical reference information and the second subset of anatomical reference information for performing navigation of the patient anatomy.

17. The navigation system of claim 16, wherein the execution of the instructions further causes the system to perform a surgical navigation of the patient anatomy using the portion of patient anatomy as registered, wherein performing the surgical navigation provides clinically relevant information for a surgery procedure.

18. The navigation system of claim 16, wherein the one or more navigational system sensors comprises an optical sensor; and, each intra-operative image is generated by an intra-operative image device that is different from the optical sensor.

19. The navigation system of claim 16, wherein the execution of the instructions further causes the system to provide any one or more of:
a relative measurement between the tool and the patient's anatomical structure;
an overlay for a portion of the tool or the cutting plane for display with an image of the patient's anatomical structure;
workflow for a procedure; or
an instruction to command a robotic manipulator.

20. A computer program product comprising a non-transitory storage device storing instructions for execution by at least one processor of a navigation system to cause the navigation system to:
receive an identification of a first subset of anatomical reference information associate with at least one intra-operative image of a surgical location, each intra-operative image having an image reference frame and each showing a same portion of patient anatomy;
register the image reference frame with a navigation system reference frame of the navigation system, wherein the navigation system is configured to track objects comprising any one or more of surgical tools and portions of patient anatomy;
receive signals, from one or more navigational system sensors, identifying a second subset of anatomical reference information, wherein at least some of the second subset of anatomical reference information are different from the first subset of anatomical reference information; and
register a portion of patient anatomy to the navigation system using at least one item of anatomical reference information from each of the first subset of anatomical reference information and the second subset of anatomical reference information for performing navigation of the patient anatomy.
